(19) United States
(12) Patent Application Publication
(10) Pub. No.: US 2026/0063516 A1
(43) Pub. Date: Mar. 5, 2026

(54) GLASS SAMPLING DEVICE

(71) Applicant: Owens-Brockway Glass Container Inc., Perrysburg, OH (US)

(72) Inventor: Kenneth Jensen, Frederick, CO (US)

(21) Appl. No.: 18/824,216

(22) Filed: Sep. 4, 2024

(51) Int. Cl. G01N 1/12; G01N 33/38

(52) U.S. Cl. CPC G01N 1/12; G01N 33/386

(57) ABSTRACT

A molten glass sampling device and method of operating the molten glass sampling device are disclosed. The molten glass sampling device includes a sampling cup and a rod. The sampling cup defines an interior cavity and is coupled to the rod, which is composed of an electrically resistive material that maintains an electrical gradient of at least 50 kV per foot for at least one minute. The molten glass sampling device can tolerate the elevated temperatures of molten glass and can handle energized molten glass. The molten glass sampling device may additionally include the added functionality of the sampling cup being movable relative to the rod.

### TECHNICAL FIELD

The present disclosure relates to a glass sampling device, and more specifically, to a molten glass sampling device having a sampling cup and a rod.

### BACKGROUND

A glass container is formed from molten glass that is produced in a glass melting apparatus such as, for example, a continuous melting furnace or a submerged combustion melter. The molten glass may be fined or refined to reduce the bubble content of the glass followed by thermally conditioning the molten glass to bring the molten glass to the correct forming viscosity and to improve its thermal homogeneity. For any of a variety of reasons-including for purposes related to melting, fining, refining, and/or conditioning-some or all of the heat added to the molten glass within the glass melting apparatus or in another structure downstream from the glass melting apparatus may be introduced directly via electrical heating. Electric heating involves passing an electrical current between electrodes that are submerged within the molten glass. Since the molten glass itself functions as a resistor, the passing electric current generates heat within the molten glass.

When forming a glass container, a discrete charge of molten glass is typically obtained from a glass feeder, which may be appended to a forehearth that supplies the glass feeder with thermally conditioned molten glass. The glass feeder advances molten glass through an exit orifice and cuts the falling glass into the glass charge of a specified weight. The molten glass charge is then delivered to a glass container forming machine where the molten glass charge is formed into the glass container. Periodically, a sample of molten glass is collected for inspection somewhere between the glass feeder and the glass container forming machine since this location is generally where glass is most accessible prior to being formed into the glass container. The collected sample may be one of the charges of discrete molten glass or it may be pulled from an uncut stream of falling molten glass. The molten glass is sampled and inspected to analyze one or more characteristics of the molten glass to support manufacturing operations including for research, data collection, and/or quality control purposes.

The sampling of molten glass that has been electrically melted or boosted upstream of the sampling point poses some practical considerations. First, the molten glass is very hot, typically having a temperature of at least 1000° C. and usually greater than 1100° C. Second, the molten glass may, in some instances, be energized and thus carry an electric charge as a byproduct of the electric heating. And third, the location where molten glass is sampled can be cramped and crowded by the presence of the glass forming machine, glass delivery equipment, utilities lines, and other operating personnel. A glass sampling device that can be used to obtain samples of molten glass that may possibly be electrically charged as result of electric heating or otherwise would help simplify the glass sampling procedure.

### SUMMARY OF THE DISCLOSURE

A molten glass sampling device includes a sampling cup and a rod coupled to the sampling cup. The sampling cup defines an interior cavity for collecting a molten glass sample and the rod is composed of an electrically resistive material to help inhibit the flow of an electric current through the rod in the event that the collected sample of molten glass is energized. The electrically resistive material that constitutes the rod can maintain an electrical gradient of at least 50 kV per foot for at least one minute and, in a preferred embodiment, comprises fiberglass. The rod may also be variable in length and be constructed so that the cup is moveably coupled to the rod. In operation, a molten glass sample, which may comprise all or part of a gob of molten glass falling from a glass feeder, is collected in the sampling cup and is later unloaded from the sampling cup for any of a variety of reasons including, for example, to analyze the glass included in the molten glass sample.

### BRIEF DESCRIPTION OF THE DRAWINGS

FIG. 1 is a perspective view of a molten glass sampling device and a schematic illustration of a portion of a glass feeder in accordance with an illustrative embodiment of the present disclosure;

FIG. 2A is a schematic side view of a molten glass sampling device in accordance with another embodiment of the present disclosure;

FIG. 2B is a schematic top view of a molten glass sampling device in accordance with yet another embodiment of the present disclosure; and

FIG. 3 is a perspective view of a molten glass sampling device in accordance with still another illustrative embodiment of the present disclosure.

### DETAILED DESCRIPTION

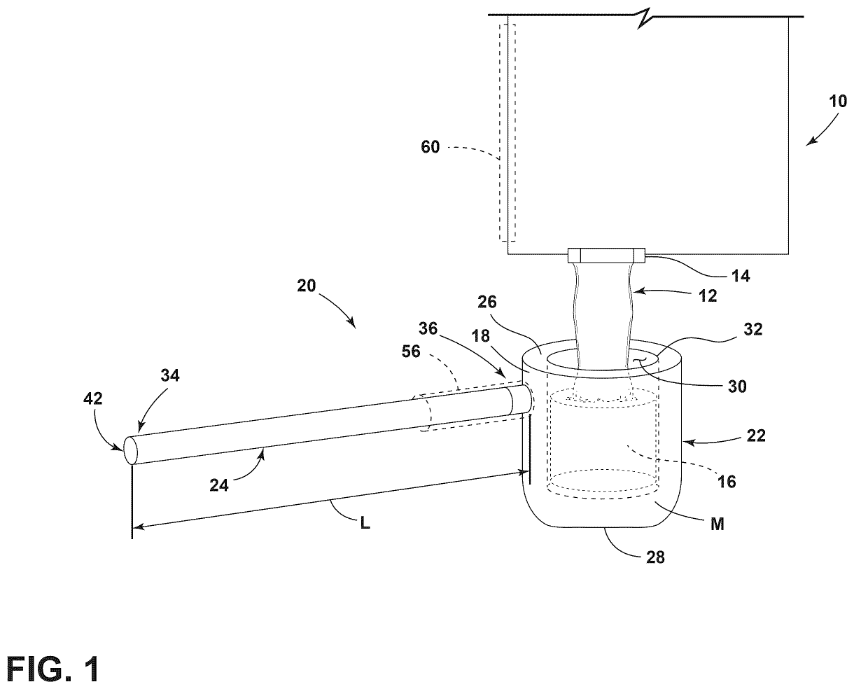

A molten glass sampling device for obtaining samples of molten glass is described in FIGS. 1-3 in the context of a glass container manufacturing setting. Ordinarily, when manufacturing a glass container, a vitrifiable feed material is first melted in a glass melting apparatus into molten glass. The glass melting apparatus may be a continuous melting furnace, a submerged combustion melter, or some other apparatus that produces molten glass. In a continuous melting furnace, the vitrifiable feed material is fed onto a molten glass bath within an upstream melting chamber of the furnace and heat is provided to the melting chamber above the glass bath in an open combustion zone by overhead burners. In contrast, in a submerged combustion melter, the vitrifiable feed material is fed into a glass melt that is agitated and heated by combustion products that are fired directly into the glass melt by submerged burners. The produced molten glass is then refined or fined, either in a refining chamber of a continuous melting furnace or in a separate finer positioned downstream of a submerged combustion melter, and delivered to a forehearth where the glass is thermally conditioned for subsequent glass forming operations.

The forehearth provides the molten glass to a glass feeder, which, in turn, modulates molten glass through an exit orifice of the feeder. The molten glass exiting the orifice is cut by shears or some other cutting device to produce a discrete charge or “gob” of molten glass. The charge of molten glass is then delivered to a glass container forming machine. For any of a variety of reasons, a sample of the molten glass may need to be taken after the molten glass exits the glass feeder or anywhere else in the plant where molten glass may be accessible. The molten glass sampling device described herein is constructed to provide robust functionality in that the device can tolerate the elevated temperatures of molten glass, which usually exceed 1000° C., and can handle energized molten glass by electrically insulating an operator from the collected molten glass sample. The molten glass sampling device may also include an articulating joint to help the device more easily obtain a sample of molten glass, especially when the sampling location is difficult to reach and/or in a crowded location.

Referring now to FIG. 1, a molten glass feeder 10 is shown with molten glass 12 being discharged through an exit orifice 14. The molten glass 12 is illustrated here as an uncut stream of falling molten glass but may also take the form of a discrete charge or gob of molten glass. The molten glass has a temperature of, for example, 1000° C. or greater and may be energized in that the glass is electrically charged. A sample 16 of the molten glass 12 may be obtained with a molten glass sampling device 20. While the molten glass 12 is depicted here as falling from a glass feeder 12, the molten glass sampling device 20 may be used in any other context or environment in which a sample of molten glass needs to be obtained.

The molten glass sampling device 20 includes a sampling cup 22 and a rod 24 coupled to the sampling cup 22. The sampling cup 22 has a first end 26 and an opposed second end 28 and defines an interior cavity 30. The first end 26 defines an opening 32 to the interior cavity 30 and the second end 28 is closed beneath the interior cavity 30. In that regard, the molten glass sample 16, which may be all or part of the molten glass 12 discharged from the glass feeder 10, may be received into the interior cavity 30 through the opening 32 and is retained in the cavity 30 until such time that the sampling cup 22 is inverted to unload molten glass sample 16 out of the interior cavity 30. To ensure the sampling cup 22 can withstand the temperature of the molten glass sample 16, at least a portion of the sampling cup 22 that surrounds and provides the interior cavity 30 is composed of a material M having a melting point of at least 1100° C. or, more preferably, at least 1600° C. By way of non-limiting example, at least this portion of the sampling cup 22 may comprise at least one of: (i) a metal such as cast iron; (ii) a ceramic such as alumina, fused silica, and/or zirconia, or (iii) graphite. The sampling cup 22 is preferably composed entirely of the material M.

The rod 24 extends from a handheld end 34 to a distal coupling end 36 and is composed of an electrically resistive material to help inhibit any electrical charge that may be carried by the molten glass sample 16 from being transferred from the sampling cup 22 to the operator of the molten glass sampling device 20. The electrically resistive material, more particularly, can maintain an electrical gradient of at least 50 kV per foot for at least one minute. The electrically resistive material may even be able to maintain an electrical gradient of at least 100 kV per foot for at least one minute or at least 100 kV per foot for at least three minutes. The procedure for determining whether a material can maintain a specified electrical gradient and for how long is set forth in ASTM F-711-17. In one specific example, the rod 24 may comprise a fiberglass shell that surrounds a foam core or remains hollow. The fiberglass shell may be a glass fiber reinforced polyester plastic. Additionally, to make the molten glass sampling device 20 more adaptable, a length L of the rod 24 between the handheld end 34 and the coupling end 36 may be variable. The rod 24, for example, may be telescopic to provide it with a variable length L.

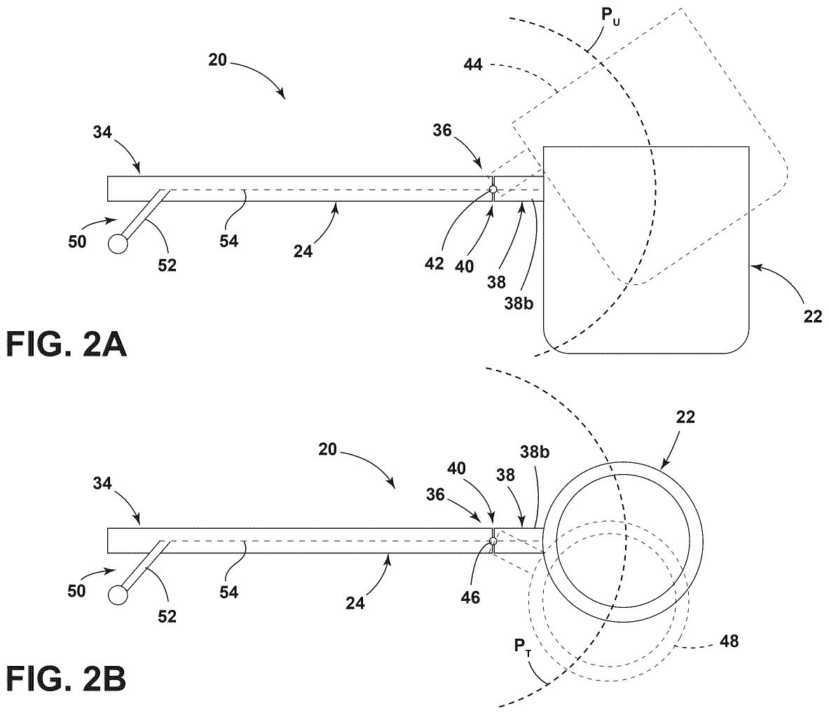

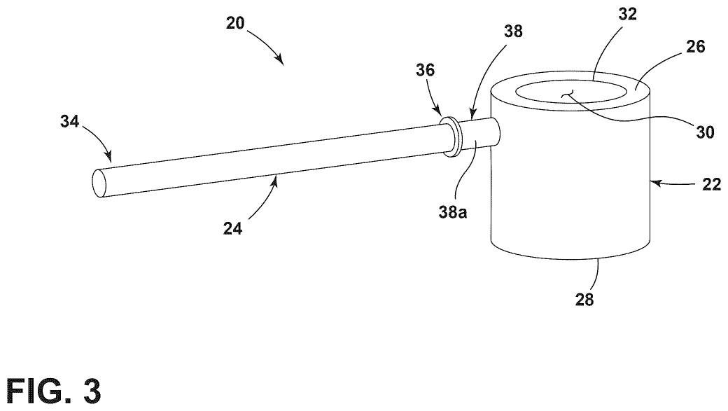

The rod 24 is coupled to the sampling cup 22 in any of a variety of ways. The rod 24 may be fixedly coupled to the sampling cup 22 such that the sampling cup is unable to move relative to the rod 24 as shown in FIG. 1. For example, the rod 24 may be permanently fixedly coupled to the sampling cup 22 by welding the coupling end 36 of the rod 24 to an exterior surface 18 of the cup 22. In another example, as shown in FIGS. 2A-3, the sampling cup 22 may include a coupling appendage 38 that projects away from the cup 22 and is coupled to the rod 24. This coupling appendage 38 may be integrally formed with the cup 22 or be attached to the cup 22. As shown in FIG. 3, for instance, the coupling appendage 38 may be a shaft 38a that extends away from the exterior of the cup 22, and the coupling end 36 of the rod 24 may include a flange that is welded to a mating flange at a free end of the shaft 38a. In other embodiments, the rod 24 may be releasably fixedly coupled to the cup 22 such that the rod 24 and the cup 22 can be decoupled without destroying the rod 24 or the cup 22. A releasable fixed coupling may be achieved by fastening together the flange on the coupling end 36 of the rod 24 and the mating flange on the shaft 38a. Or, alternately, the shaft 38a may be an internally-threaded sleeve, and a releasable fixed coupling may be achieved by threading an externally-threaded portion of the coupling end 36 of the rod 24 into the internally-threaded sleeve.

The rod 24 may also be movably coupled to the sampling cup 22 by an articulating joint 40 that permits the cup 22 to move relative to the rod 24 in at least one direction. For example, and as shown in FIG. 2A, the articulating joint 40 may include a pivot joint that permits the sampling cup 22 to pivot about a tilt axis 42 and thus rotate bidirectionally along an upright arcuate path Pu. The pivot joint may include an axis pin that is received by aligned bushings, bearings, knuckles, or holes on the coupling end 36 of the rod 24 and the coupling appendage 38, which, here, is a shaft 38b either in solid or hollow form. When pivoting about the tilt axis 42, the sampling cup 22 can be tilted upwards in one direction (dashed lines 44) along the upright arcuate path Pu to angle the opening 32 of the cup 22 towards the rod 24 and tilted downwards in an opposite direction along the upright arcuate path Pu to angle the opening of the cup 22 away from the rod 24. In another example, as shown in FIG. 2B, the articulating joint 40 may include a pivot joint that permits the sampling cup 22 to pivot about a hinge axis 46 and thus rotate bidirectionally along a transverse arcuate path Pr. When pivoting about the hinge axis 46, the sampling cup 22 can be swung crosswise in one direction (dashed lines 48) along the transverse arcuate path PT to bring the cup 22 to one side of the rod 24 and swung crosswise in an opposite direction along the transverse arcuate path Pr to bring the cup 22 to an opposite side of the rod 24.

The articulating joint 40 is not limited to fostering pivoting rotation of the sampling cup 22 along only a single arcuate path. In each of FIGS. 2A and 2B, the articulating joint 40 allows the sampling cup 22 to rotate in two directions along a single arcuate path Pu, PT. While such movement may be sufficient, pivoting rotation along multiple arcuate paths or some other form of movement is certainly possible. For instance, the articulating joint 40 may include two pivot joints such that the sampling cup 22 can pivot about both the tilt axis 42 and the hinge axis 46, thus allowing the cup 22 to rotate in four directions. In still another example, the articulating joint 40 may permit the sampling cup 22 to move in three-dimensions relative to the rod 24. To allow for such movement, the coupling end 36 of the rod 24 may include a ball that is received into a socket defined in the coupling appendage 38, thereby creating a ball-and-socket joint. Of course, with any of the joints described above that form all or part of the articulating joint 40, the joint may be permanent or releasable.

At least one cup movement control 50 may be carried by the rod 24. The cup movement control 50 is operative to selective move the sampling cup 22 relative to the rod 24. For example, and referring to the embodiments illustrated in FIGS. 2A-2B, the cup movement control 50 may be operable to move the sampling cup 22 about the tilt axis 42, about the hinge axis 46, about both axes 42, 46, or in other directions such as in three-dimensions relative to the rod 24. The cup movement control 50 includes an actuator 52 and a linkage 54. The actuator 52 is carried on the rod 24 proximate the handheld end 34 of the rod 24, meaning the actuator is located closer to the handheld end 34 than to the coupling end 36. The actuator 52 may be a lever, as shown, or any of a wide variety of other operable actuating devices including a depressible button, a squeezable handle, a pullable handle, a rotatable crank handle, and/or a hand knob. The linkage 54 is operably connected to the actuator 52 and to the sampling cup 22. The linkage 54 may be an elongated connecting bar, an elongated screw, one or more springs or bands, a reverse-motion linkage, a push-pull linkage, or any other connecting device that can effectuate relative movement of the sampling cup 22 in response to operation of the actuator 52. While the cup movement control(s) 50 may be convenient, such control(s) 50 are not mandatory as the cup 22 may be moved relative to the rod 24 manually by grabbing the cup 22 and adjusting its position.

The molten glass sampling device 20 may additionally, and optionally, include thermal insulation 56 that covers at least a portion of the sampling cup 22, at least a portion of the rod 24, or at least a portion of the sampling cup 22 and the rod 24, as shown in FIG. 1. The thermal insulation 56 helps protect the sampling cup 22 and the rod 24 from exposure to heat and molten glass. Indeed, when collecting the molten glass sample 16, the molten glass sampling device 20 and, in particular, the sampling cup and the coupling end 36 of the rod 24, may be positioned in close proximity to hot molten glass. Molten glass may even make contact with the sampling cup 22 outside of the interior cavity 30 and/or the rod 24. The thermal insulation 56 acts as a protective barrier to such exposures and may be a woven tape, a wrapped fabric and/or an applied coating. In one embodiment, the thermal insulation 56 covers at least a portion of the rod 22 including the coupling end 36 of the rod 24 and may even cover the entire rod 24 up to the cup movement control(s) 50.

A method of using the molten glass sampling device 20 includes a step of providing the molten glass sampling device 20 and a step of collecting a molten glass sample 16 with the device 20. The step of providing the glass sampling device 20 involves simply obtaining the device 20 in its constructed form. The step of collecting the molten glass sample 16 may be performed in various ways depending on where and how the molten glass sample 16 needs to be collected. For example, when collecting the molten glass sample 16 from molten glass 12 that is falling, such as from the glass feeder 10, the sampling device 20 is maneuvered to position the sampling cup 22 at a collection location under the feeder 10 and within the path of the molten glass 12 that is falling. Some or all of the falling molten glass 12 is then received into the interior cavity 30 through the opening 32 and is retained within the interior cavity 30 as the molten glass sample 16. In another embodiment, the molten glass sample 16 may be collected by scooping the sample 16 out of a larger volume of glass after, for instance, inserting the sampling cup 22 through a window 60 (FIG. 1) of the glass feeder 10 or some other vessel that contains the volume of molten glass. The molten glass sample 16 may be energized.

The method may also include a step of unloading the molten glass sample 16 and a step of moving the sampling cup 22 relative to the rod 24. The step of unloading the molten glass sample 16 occurs after collecting the molten glass sample 16 and, in the scenario in which the molten glass sample 16 is obtained from molten glass 16 that is falling, involves maneuvering the molten glass sampling device 20 to position the sampling cup 22 away from the collection location followed by unloading the molten glass sample 16 from the interior cavity 30 for analysis. The molten glass sample 16 may be unloaded by inverting the sampling cup 22 so that the molten glass sample 16 falls out of the interior cavity 30. The step of moving the sampling cup 22 relative to the rod 24 may occur before, during, or after the step of collecting the molten glass sample 16; to be sure, the sampling cup 22 may be moved relative to the rod 24 before, during, or after receipt of the molten glass sample 16 into the interior cavity 30. The sampling cup 22 may be moved relative to the rod 24 by engaging the at least one cup movement control 50—such as, for example, by operating the actuator 52 to cause a response in the linkage 54 that operatively connects the actuator 52 to the cup 22—to induce movement at the articulating joint 40 and a corresponding selectively movement of the cup 22 relative to the rod 24.

The subject matter of this application is presently disclosed in conjunction with several explicit illustrative embodiments and modifications to those embodiments, using various terms. All terms used herein are intended to be merely descriptive, rather than necessarily limiting, and are to be interpreted and construed in accordance with their ordinary and customary meaning in the art, unless used in a context that requires a different interpretation. As such, many other embodiments, modifications, and equivalents thereto, either exist now or are yet to be discovered and, thus, it is neither intended nor possible to presently describe all such subject matter, which will readily be suggested to persons of ordinary skill in the art in view of the present disclosure. Rather, the present disclosure is intended to embrace all such embodiments and modifications of the subject matter of this application, and equivalents thereto, as fall within the broad scope of the accompanying claims.

1. A molten glass sampling device comprising:
a sampling cup having a first end and an opposed second end and defining an interior cavity, the first end of the sampling cup defining an opening to the interior cavity and the second end of the sampling cup being closed; and
a rod coupled to the sampling cup, the rod being composed of an electrically resistive material that maintains an electrical gradient of at least 50 kV per foot for at least one minute.

2. The molten glass sampling device set forth in claim 1, wherein the electrically resistive material maintains an electrical gradient of at least 100 kV per foot for at least three minutes.

3. The molten glass sampling device set forth in claim 1, wherein a length of the rod is variable.

4. The molten glass sampling device set forth in claim 1, wherein the rod is removably coupled to the sampling cup.

5. The molten glass sampling device set forth in claim 1, wherein the sampling cup is moveably coupled to the rod such that the sampling cup is moveable relative to the rod.

6. The molten glass sampling device set forth in claim 5, wherein the sampling cup is rotatable at least about a hinge axis.

7. The molten glass sampling device set forth in claim 5, wherein the sampling cup is rotatable at least about a tilt axis.

8. The molten glass sampling device set forth in claim 5, wherein the sampling cup is rotatable about both a hinge axis and a tilt axis.

9. The molten glass sampling device set forth in claim 5, wherein the sampling cup is movable in three dimensions relative to the rod.

10. The molten glass sampling device set forth in claim 1, wherein the rod comprises fiberglass.

11. The molten glass sampling device set forth in claim 1, wherein at least a portion of the sampling cup that defines the interior cavity is comprised of a material having a melting point of at least 1100° C.

12. The molten glass sampling device set forth in claim 11, wherein the portion of the sampling cup is comprised of at least one of a metal, a ceramic, or graphite.

13. The molten glass sampling device set forth in claim 1, wherein the sampling cup includes a coupling appendage that protrudes away from the cup and is coupled to the rod.

14. The molten glass sampling device set forth in claim 13, wherein the coupling appendage is a shaft.

15. A method of operating a molten glass sampling device, the method comprising:
providing the molten glass sampling device that includes a sampling cup and a rod coupled to the sampling cup, the sampling cup having a first end and an opposed second end and defining an interior cavity with the first end of the sampling cup defining an opening to the interior cavity and the second end of the sampling cup being closed, and the rod being composed of an electrically resistive material that maintains an electrical gradient of at least 50 kV per foot for at least one minute;
collecting a molten glass sample in the interior cavity of the sampling cup; and
unloading the molten glass sample from the sampling cup.

16. The method set forth in claim 15, further comprising:
moving the sampling cup relative to the rod.
